(19) United States
(12) Patent Application Publication
(10) Pub. No.: US 2023/0383238 A1
(43) Pub. Date: Nov. 30, 2023

(54) METHODS FOR PROCESSING AND ANALYZING CELL-DERIVED VESICLES

(71) Applicants: Capsugel Italy S.R.L., Milan (IT); MDimune Inc., Seoul (KR)

(72) Inventors: Davide ZOCCO, Milan (IT); Ilaria PASSALACQUA, Milan (IT); Shin Gyu BAE, Seoul (KR); Seung Wook OH, Mukilteo, WA (US); Jeong Eun PARK, Seoul (KR); Sung-Soo PARK, Seoul (KR); Hui-Chong LAU, Seoul (KR)

(21) Appl. No.: 17/936,027

(22) Filed: Sep. 28, 2022

Related U.S. Application Data
(60) Provisional application No. 63/345,143, filed on May 24, 2022.

(51) Int. Cl. C12M 1/00; C12N 5/0735; C12M 1/26

(52) U.S. Cl. CPC C12M 47/02; C12N 5/0606; C12M 45/05; C12M 33/14

(57) ABSTRACT

The present disclosure provides methods for processing cell-derived vesicles in which the cell-derived vesicles are not purified, prior to contacting with a fluorescent staining dye or an antibody. By utilizing a centrifugal filter, excess staining dye or antibody can be readily removed prior to analysis of one or more characteristics of the cell-derived vesicles. The methods provide rapid and simple processing and analysis, while maintaining a high concentration of cell-derived vesicles.

### CROSS-REFERENCE TO RELATED APPLICATIONS

The present application claims benefit of U.S. Provisional Application No. 63/345,143, filed May 2, 2022, the disclosure of which is incorporated by reference herein in its entirety.

### FIELD OF THE INVENTION

The present disclosure provides methods for processing cell-derived vesicles (CDVs). By utilizing a centrifugal filter, excess staining dye or antibody can be readily removed prior to analysis of one or more characteristics of the CDVs. The methods provide rapid and simple processing and analysis, while maintaining a high concentration of CDVs.

### BACKGROUND OF THE INVENTION

Research into the applications and treatments using exosomes, or extracellular vesicles, for various cancers and other conditions continue to develop. The ability of these 50-150 nm cell-derived vesicles to deliver various cargo, include proteins, lipids and nucleic acids (including siRNA and antisense nucleic acids), has lead to interest in utilizing them for delivery to various different cell types.

An alternative to exosomes is the production of cell-derived vesicles (CDVs) from nucleated mammalian cells. CDVs are exosome-mimetic nanovesicles prepared from serial extrusion of nucleated cells through filters with diminishing pore size. The resulting CDVs exhibit many similarities to exosomes in terms of size, morphology, and the molecular composition of membranes, but have a 100-fold higher production yield. CDVs can also be produced from any nucleated mammalian cell type, and can be loaded with various therapeutic agents and effectively delivered to target cells and tissues.

In order to isolate and analyze vesicles such as CDVs generated by extrusion from a given cell population, various methods have been developed. However, these traditional approaches often result in loss of CDVs, or significantly dilute the sample, which can lead to inconsistent or compromised analysis. Therefore, what is needed is a simple, rapid process that provides for CDV separation and analysis, without unwanted dilution. The present invention provides such processes.

### SUMMARY OF THE INVENTION

In embodiments, provided herein is a method for processing cell-derived vesicles (CDVs), comprising: concentrating CDVs in a biological fluid; determining a concentration of the CDVs; contacting the CDVs with a fluorescent staining dye or an antibody for an CDV surface marker; incubating the contacted CDVs to generate a labeled CDV population; passing the contacted CDVs through a centrifugal filter comprising a 200-500 kD molecular weight cut off, polyethersulfone filter media, to separate the labeled CDV population from excess fluorescent staining dye or excess antibody; and recovering the labeled CDV population.

In further embodiments, provided herein is a method for analyzing CDVs, comprising: concentrating CDVs in a sample with a tangential flow filter; determining a concentration of the CDVs; contacting the CDVs with a fluorescent staining dye or an antibody for a CDV surface marker; incubating the contacted CDVs to generate a labeled CDV population; passing the contacted CDVs through a centrifugal filter comprising a 300 kD molecular weight cut off, polyethersulfone filter media, to separate the labeled CDV population from excess fluorescent staining dye or excess antibody; recovering the labeled CDV population; and analyzing the recovered, labeled CDV population using a flow cytometer for nanoparticle analysis.

### BRIEF DESCRIPTION OF THE DRAWINGS

FIG. 1 shows relative amounts of CDV markers. * SCARB2: lysosome membrane protein 2; LAMP1: lysosome-associated membrane glycoprotein 1 LAMP2: lysosome-associated membrane glycoprotein 2; NCSTN: nicastrin; RAB7A: Ras-related protein Rab-7a; KTN1: kinectin; ATP1B3: sodium/potassium transporting ATPase subunit beta-3; BSG: basigin; ITGB1: integrin beta-1.

FIGS. 2A-2C show (A) Western blotting analyses; (B) nanoparticle flow cytometry results; and (C) CFSE staining.

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps.

In embodiments, provided herein is a method for processing cell-derived vesicles. The term “cell-derived vesicles” (CDVs) as used herein refers to vesicles artificially synthesized by serial extrusion from nucleated (i.e., cells that contain a nucleus) mammalian cells. The lipid of the cell membranes forms the CDVs, defining their internal space thereof from the external environment. The CDVs disclosed herein have membrane proteins, nucleic acids and cellular components from the parent cells in addition to the membrane lipids. The CDVs can also be engineered to carry cargo such as nucleic acids, proteins, peptides, and drugs. CDVs are suitably on the order of about 100 nm to about 400 nm in size, more suitably about 100 nm to about 300 nm, about 100 nm to about 250 nm, about 150 nm to about 250 nm, about 100 nm, about 150 nm, about 200 nm, about 250 nm or about 300 nm, in size.

The processing methods described herein are used to separate CDVs from a biological fluid or sample, following their production from one or more cell types. As used herein a “biological fluid” or “sample” refers to a solution that suitably comprises cells, cellular debris, buffers, cell growth media, etc., that is used in the production of CDVs. The “biological fluid” or “sample” can be any growth media, buffer, or solution that comprises CDVs obtained from nucleated cells.

In embodiments, the CDVs are produced by a method which comprises preparing a suspension of nucleated mammalian cells and conducting serial extrusion of the nucleated cells by sequentially passing the cells through filters with diminishing micro-size pores to produce a biological fluid or a sample comprising CDVs. Such a method is described in U.S. Pat. No. 10,675,244, the disclosure of which is incorporated by reference herein in its entirety, in particular the methods of preparing CDVs described therein. In embodiments, the suspension of cells is sequentially passed through each filter one time, two times, three times, four times, or more. In embodiments, the first filter has a pore size of 7, 8, 9, 10, 11, or 12 μm. In embodiments, the second filter has a pore size of 1, 2, 3, 4, 5, or 6 μm. In embodiments, the third filter has a pore size of 0.1, 0.2, 0.3, 0.4, 0.5, 0.6, 0.7, 0.8, 0.9, or 1 μm. In some embodiments, the first filter has a pore size of 10 μm, the second filter has a pore size of 5 μm, and the third filter has pore size of 1 μM. In some embodiments, the first filter has a pore size of 10 μm, the second filter has a pore size of 3 μm, and the third filter has a pore size of about 0.4 μm.

Any nucleated mammalian cell type can be used to produce CDVs, as is known in the art. In exemplary embodiments, the CDVs are produced from human embryonic kidney (HEK) cells (including HEK-293 cells), Human Caucasian colon adenocarcinoma HT29 cells, or mesenchymal stem cells (MSCs). In some embodiments, the cells that can be used to prepare the CDVs include, but are not limited to, embryonic stem cells and cells derived from embryonic stem cells, induced pluripotent stem cells, endothelial progenitor cells, immature and mature dendritic cells (DC), monocytes, macrophages, T and B lymphocytes, fibroblasts, epithelial cells, endothelial cells, including human umbilical vein endothelial cells (HUVEC), myocytes, cardiomyocytes, neuronal cells, glial cells, kidney cells, pancreatic cells, stromal cells, keratinocytes, or melanocytes. In further embodiments, the CDVs can be produced from various disease cell lines, including various cancer cells lines. In some embodiments, the cells are isolated from primary cell cultures. In other embodiments, the cells are cell lines. In embodiments, the methods described herein are useful for analyzing disease characteristics present in CDVs obtained from various cell types, such as healthy and cancer cells.

In embodiments, the cells for producing CDVs express proteins which are naturally expressed in the cytoplasm or on the cell membrane of the cells. In some embodiments, the cells for producing CDVs are transformed such that the expression of these proteins is upregulated or downregulated. In other embodiments, the cells for producing CDVs are transformed such that they express a protein which is not normally expressed by that cell type. In some embodiments, the expression of a specific protein or proteins of interest is either upregulated or downregulated by transformation of the cells. The transformation of cells can be achieved using typical methods known in the art, for example, by stimulating the cells or introducing foreign genes into the cells to modify, e.g., upregulate or downregulate the expression of proteins of interest. A specific stimulus may induce a change in the expression of proteins of interest. For example, when treated with TNF-α, human umbilical vein endothelial cells (HUVEC) overexpress ICAM-1 in the plasma membrane [J. Exp. Med. 177; 1277-1286 (1993)]. In monocytes treated with PMA (phorbol 12-myristate 13-acetate), the membrane protein LFA-1 is activated [J. Exp. Med. 163; 1132-1149 (1986)]. The introduction of foreign genes may induce the expression or inhibition of proteins of interest. In this context, plasmid DNA, RNA or virus is introduced into cells [PNAS. 90 (18); 8392-8396 (1993)] using calcium phosphate precipitation [Current Protocols in Cell Biology 20.3.1-20.3.8 (2003)], lipofectamine mediation [PNAS. 84 (21); 7413-7417 (1987)], electroporation [Nucleic Acids Research. 15 (3) 1311-1326 (1987)], microinjection [Mol Cell Biol. 2(9); 1145-1154 (1982)], ultrasound mediation [Human Gene Therapy. 7(11); 1339-1346 (1996)] or other methods known in the art. Various gene editing methods, including CRISPR, TALENS and recombinational cloning methods can also be used to modify or introduce various genes into a cell, as desired.

In embodiments, the cells for producing CDVs are induced to express one or more receptors or ligands for one or more proteins of interest, and the CDVs produced from these cells display the one or more receptors or ligands on their surface. In other embodiments, the cells for producing CDVs are induced to express one or more antibodies, and the CDVs produced from these cells display the one or more antibodies on their surface. In embodiments, the antibodies are specific for a protein expressed on a target cell of interest and can include, but are not limited to, antibodies which bind normal cell markers or tumor-associated antigens. In further embodiments, the CDVs produced from these cells express a T cell receptor. In embodiments, the TCR can be a naturally occurring T cell receptor or can be a recombinant and/or chimeric T cell receptor. In such embodiments, the T cell receptor can bind a normal cell maker or a tumor-associated antigen.

In some embodiments, the cells for producing CDVs are induced to express a protein, peptide, or nucleic acid within the cells or displayed on the CDV surface. Examples of such proteins or peptides include, but are not limited to growth factors, such as vascular endothelial growth factor (VEGF), epidermal growth factor (EGF), human growth factor (hGF), fibroblast growth factor (FGF), cytokines, interleukins, interferons, antibodies, T cell receptors, Fc proteins, Fc receptors, immune check-point proteins and ligands thereof, including PD-1, PD-L1, PD-L2, CD27, CD28, CD40, CD122, CD137, OX40, OX40L, GITR, ICOS, and CTLA4, and fluorescent protein markers. In other embodiments, the nucleic acids expressed by the cells for producing CDVs can include, but are not limited to, DNA, RNA, mRNA, miRNA, siRNA, antisense RNA, and sense RNA.

Cells for producing CDVs can be isolated from a mammalian subject in the form of a tissue biopsy or tissue sample, and cultured and/or expanded according to methods known in the art. In other embodiments, the cells for producing CDVs are cell lines that are produced in a bioreactor prior to use in the methods of processing described herein. The cells can be prepared in any suitable bioreactor (also called reactor herein) including but not limited to stirred tank, airlift, fiber, microfiber, hollow fiber, ceramic matrix, fluidized bed, fixed bed, and/or spouted bed bioreactors. As used herein, “bioreactor” can include a fermenter or fermentation unit, or any other reaction vessel and the terms “bioreactor” and “reactor” are used interchangeably with “fermenter.” The term fermenter or fermentation refers to both microbial and mammalian cultures. For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments, the CDVs processed and/or analyzed by the methods disclosed herein may be loaded with a therapeutic and/or diagnostic substance. In some embodiments, the CDVs can be produced from a cell which has already been loaded with therapeutic and/or diagnostic substances of interest. For example, when cells are cultured in a medium containing the therapeutic and/or diagnostic substances of interest, they may contain the substances therein. Alternatively, the substances may be introduced into cells by electroporation. CDVs produced from the cells by extrusion are thus are loaded with the substances.

In other embodiments, the therapeutic and/or diagnostic substances may be loaded into CDVs in the course of the construction thereof. For instance, when a cell suspension containing substances of interest is extruded through sub-cell size filters, the CDVs thus formed are loaded with the substances. In further embodiments, CDVs may be loaded with substances of interest after they are produced by cell extrusion. For example, the loading of therapeutic and/or diagnostic substances can be achieved by incubating a suspension of CDVs with the substances or by electroporating the substances into already prepared CDVs. However, it should be appreciated to those skilled in the art that the loading of substances of interest into CDVs is not limited to the above-illustrated methods.

In embodiments, the therapeutic and/or diagnostic agents include, but are not limited to, anticancer agents, anti-inflammatory agents, angiogenesis inhibitors, peptides, proteins, toxins, nucleic acids, beads, microparticles, and nanoparticles.

In embodiments, the methods for processing CDVs include concentrating CDVs obtained by extrusion from nucleated cells in a biological fluid or a sample, i.e., concentrating CDVs that are present in a biological fluid or a sample. Methods of concentrating CDVs include for example, passing the CDVs through one or more tangential flow filters to concentrate the CDVs (i.e., decrease the fluid volume while maintaining the number of CDVs in the sample). Tangential flow filtration, also known as crossflow filtration, is a filtration system or process where a feed, inlet or input fluid stream passes parallel to a membrane face as one portion passes through, and out of the membrane (permeate flow) while the remainder (retentate flow) passes within the membrane and can be recirculated back to the input, becomes concentrated, can ultimately be passed to storage or for further processing. A tangential flow filter is suitably comprised of a series of hollow fiber membranes (though a single fiber can also be used), into which a solution is fed. The retentate flow passes within the hollow fiber, retaining CDVs within the solution inside of the fiber membrane, while excess volume passes through the fiber membrane and out into the permeate flow. This reduces the volume of the total sample, resulting in a concentrating of the CDV sample (an increase in number of CDVs per volume). Exemplary materials for use in a tangential flow filter include polymers, including but not limited to, poly(ether sulfone), poly(acrylonitrile) and poly(vinylidene difluoride), cellulose esters, and poly(sulfone). Exemplary tangential flow filters include those available from SPECTRUM LABS® or REPLIGEN®, including MICROKROS® and MIDIKROS® filters, and modifications thereof.

In exemplary embodiments, the CDVs are concentrated by first passing the CDVs through a tangential flow filter having a molecular weight cut-off of between about 200 kD and about 750 kD, suitably about 300 kD to about 750 kD, about 400 kD to about 750 kD, about 500 kD to about 750 kD, about 600 kD to about 750 kD, or about 500 kD, about 600 kD, about 700 kD, about 750 kD or about 800 kD. In exemplary embodiments, the tangential flow filter is a SPECTRUM® 750 kD filter from Repligen, US. In addition the passing the CDVs through a tangential flow filter, the concentrating can further include purification via size exclusion chromatography, and then further concentrated via a 3 kD filter.

The CDVs can also be processed through one or more centrifugation steps, such as 300×g for about 10 minutes, followed by 1200×g for about 20 minutes, followed by 10,000×g for about 30 min. Additional centrifugation steps can also be used. In addition, the speed and duration of centrifugation can be modified, for example, to between about 200×g-500×g for about 5-20 minutes, followed by about 800×g-1500×g for about 10-30 minutes, followed by about 7,000×g-15,000×g for about 20-40 minutes.

The methods of processing further comprise determining a concentration of the CDVs. Various methods are known in the art for determining the concentration of CDVs, and include for example, dynamic light scattering, flow cytometry for nanoparticle analysis (nanoscale flow cytometry) (e.g., NanoFCM (Nottingham, UK), and nanoparticle tracking analysis (Nanosight Instruments, Malvern Instruments; ViewSizer, Horiba), etc.

As described herein, suitably the concentration of CDVs is determined to be at least about 0.5×10¹⁰ CDVs/mL, prior to continuing the processing methods. More suitably, the concentration of CDVs is determined to be at least 1×10¹⁰ CDVs/mL, prior to continuing the processing methods, more suitably at least 0.8×10¹⁰, at least 0.9×10¹⁰, at least 1.1×10¹⁰, at least 1.2×10¹⁰, at least 1.3×10¹⁰, at least 1.4×10¹⁰, or at least 1.5×10¹⁰. As described herein, it has been determined that by achieving a concentration of CDVs of about at least 1×10¹⁰, the remainder of the labeling, cleaning/separating/washing elements of the process, and ultimate analysis of the CDVs, can be carried out reproducibly and with reduced overall waste.

As described herein, the methods further comprise contacting the CDVs with a fluorescent staining dye or an antibody for an CDV surface marker. The contacted CDVs are then incubated to generate a labeled CDV population. In suitable embodiments, the CDVs are contacted with a fluorescent staining dye that permeates the membrane of the CDVs and stains one or more intra-CDV molecules. For example, the fluorescent staining dye can be carboxyfluorescein succinimidyl ester (6-Carboxyfluorescein succinimidyl ester; 5(6)-CFDA-SE) (CFSE), a dye that couples, via its succinimidyl group, to intra-CDV molecules, notably, to intracellular lysine residues and other amine sources. Additional dyes, including fluorescent staining dyes, that can be used to label the CDVs include, for example, ExoBrite™ EV membrane stain (Biotium, Fremont, CA), ExoGlow™ EV stain (System Biosciences, Palo Alto, CA), as well as membrane dyes such as PKH67 (Sigma Aldrich). Dyes that stain RNA can also be utilized. For example, RNA staining dyes such as SYTO™ RNASelect™ and Quant-iT™ RiboGreen™ Additional dyes are also known in the art and can be used in the described methods as well.

Suitably, the CDVs are contacted with the fluorescent staining dye and incubated for at least 1 hour at a temperature of about 30° C. to 40° C. For example, the CDVs can be contacted with the fluorescent staining dye for about 30 minutes to about 2 hours, or about 30 minutes to about 1.5 hours, or about 45 minutes to about 1.5 hours, or about 1 hour to about 1.5 hours, or about 1.5 hours, at a temperature of about 35° C. to 40° C., or about 37° C.

In methods in which the CDVs are labeled with antibodies, one or more antibodies can be selected for a specific CDV surface marker. In some embodiments, the CDV surface marker is a protein naturally expressed by the cell from which the CDV was produced. In other embodiments, the CDV surface marker is a protein that was upregulated or downregulated on the surface of the cell from which the CDV was produced. That is, a surface marker that is expected to be on the surface of CDVs, or desired to be on the surface of CDVs that contain a wanted cargo (e.g., protein, peptide, nucleic acid, etc.). In exemplary embodiments, the antibodies are anti-tetraspanin antibodies, that is antibodies that bind to tetraspanin glycoproteins on the surface of the CDVs. Tetraspanins are small membrane proteins (200-350 amino acids), which interact laterally with multiple partner proteins and with each other to form the so-called TEMs (tetraspanin-enriched microdomains). Exemplary antibodies, include, but are not limited to, an anti-CD9 antibody, an anti-CD63 antibody, an anti-CD81 antibody, and an anti-IgG1 antibody. Additional antibodies can include an anti-CD151 antibody, an anti-CD82 antibody, an anti-CD53 antibody, an anti-CD37 antibody, etc. Suitably, the CDVs are labeled with combinations of such antibodies, such as a combination of an anti-CD9 antibody, an anti-CD63 antibody, an anti-CD81 antibody, and an anti-IgG1 antibody. For example, (CD9+CD63; CD9+CD81; CD81+CD63) and triple (CD9+CD81+CD63) combinations can be used.

In some embodiments, the CDVs are labeled with antibodies which are specific for a cell surface marker, a growth factor, cytokine, interleukin, interferon, or receptor thereof.

Suitably, the CDVs are contacted with the antibody(ies) and incubated for at least 30 minutes at a temperature of about 30° C. to 40° C. For example, the CDVs can be contacted with the antibodies for about 30 minutes to about 2 hours, or about 30 minutes to about 1.5 hours, or about 45 minutes to about 1.5 hours, or about 1 hour to about 1.5 hours, or about 1 hour, at a temperature of about 35° C. to 40° C., or about 37° C.

Following the labeling, the CDVs (including both labelled as well as unlabeled CDVs) are passed through a centrifugal filter comprising a 200-750 kD molecular weight cut off, polyethersulfone filter media, to separate the labeled CDV population from excess fluorescent staining dye or excess antibody. The labeled CDV population is then recovered.

As described herein, it has been surprisingly found that by passing the labeled CDV population through a polyethersulfone filter with a cutoff of about 200-750 kD molecular weight, labeled CDVs are recovered at a very high number without significant loss of CDVs, and without significant dilution of the CDVs. In suitable embodiments, the contacted CDVs (labeled with dye or antibodies) are passed through the centrifugal filter for at least 10 minutes at a centrifugal force of at least 10,000×g. Suitably, the molecular weight cutoff of the polyethersulfone filter is about 200-500 kD, about 200-400 kD, or 200 kD, 300 kD, 400 kD or 500 kD. An exemplary 300 kD molecular weight cut-off filter is a NANOSEP® centrifugal filter with OMEGA™ 300K polyethersulfone membrane from PALL® Corporation (Port Washington, NY).

In further embodiments, provided herein is a method for analyzing CDVs. In exemplary embodiments, such methods comprise concentrating CDVs in a biological fluid or sample with a tangential flow filter; determining a concentration of the CDVs; contacting the CDVs with a fluorescent staining dye or an antibody for a CDV surface marker; incubating the contacted CDVs to generate a labeled CDV population; passing the contacted CDVs through a centrifugal filter comprising a 300 kD molecular weight cut off, polyethersulfone filter media, to separate the labeled CDV population from excess fluorescent staining dye or excess antibody; recovering the labeled CDV population; and analyzing the recovered, labeled CDV population using a flow cytometer for nanoparticle analysis.

The methods of analysis described herein allow for the determination of one or more of labeling efficiency, CDV number, CDV concentration, CDV protein expression, and CDV size. Other analytical techniques, beyond the use of a flow cytometer can also be used, including for example, various fluorescent microscopy techniques, liquid chromatography techniques, mass spectrometry, NMR spectroscopy, microfluidic resistive pulse sensing (MRPS), etc. The methods of analysis described herein can suitably be used as part of a manufacturing process for a quality control check of the CDVs during production. Such methods allow for the rapid and easy determination if the methods are producing the desired CDVs, so that further manufacturing can be continued, or modified as needed, or halted, due to undesired CDVs or CDV characteristics.

As described herein, in exemplary embodiments, the CDVs are contacted with the fluorescent staining dye 6-Carboxyfluorescein succinimidyl ester (CFSE), and suitably incubated for at least 1 hour at a temperature of about 30° C.-40° C.

In embodiments where the CDVs are contacted with antibodies, suitably one or more of an anti-CD9 antibody, an anti-CD63 antibody, an anti-CD81 antibody, and/or an anti-IgG1 antibody, are utilized. Suitably, the CDVs are contacted with the antibody and incubated for at least 30 minutes at a temperature of about 30° C.-40° C.

Various cell populations can be utilized to prepare the CDVs. As described herein, suitably the CDVs are produced from human embryonic kidney (HEK) cells, Human Caucasian colon adenocarcinoma HT29 cells, or mesenchymal stem cells (MSCs). In some embodiments, the cells that can be used to prepare the CDVs include, but are not limited to, embryonic stem cells and cells derived from embryonic stem cells, induced pluripotent stem cells, endothelial progenitor cells, immature and mature dendritic cells (DC), monocytes, macrophages, T and B lymphocytes, fibroblasts, epithelial cells, endothelial cells, including human umbilical vein endothelial cells (HUVEC), myocytes, cardiomyocytes, neuronal cells, glial cells, kidney cells, pancreatic cells, stromal cells, keratinocytes, or melanocytes. In further embodiments, the CDVs can produced from various disease cell lines, including various cancer cells lines. In some embodiments, the cells are isolated from primary cell cultures. In other embodiments, the cells are cell lines. In embodiments, the methods described herein are useful for analyzing disease characteristics present in CDVs obtained from various cell types, such as healthy and cancer cells.

As described herein, it has been surprisingly found that the contacted CDVs can passed through a centrifugal filter (comprising a 300 kD molecular weight cut off, polyethersulfone filter media) for at least 10 minutes at a centrifugal force of at least 10,000×g, to separate the CDVs, while still maintaining a high concentration of the CDVs for analysis, and without losing a significant number of the CDVs during the filtration process.

### EXAMPLES

### Introduction

As described herein, cell-derived vesicles (CDVs) can be obtained from virtually any cell by using the disclosed serial extrusion technology. Similarities and differences exist between CDVs and exosomes. Particularly, among well-known exosome markers, CD9 and CD81 are less represented in CDVs compared to exosomes while CD63 is more prominent in CDVs. At the single-particle level, three tetraspanin markers are differentially represented between CDVs and exosomes. A systematic survey of marker expression profiles to better understand CDVs and their therapeutic potentials is described herein.

### Methods

Multiple batches of CDVs were produced from HEK293 cells by extruding cells serially through membrane filters (10, 3, and 0.4 μm). Exosomes were obtained from the culture medium of the same cell sources. Then, both CDVs and exosomes were purified using the same purification processes as described herein to minimize artifacts associated with purification methods. Briefly, CDVs or exosomes were processed by Tangential Flow Filtration (TFF) with 750 kDa hollow fiber column filters (Repligen, US). After TFF, the CDV and exosomes were centrifuged for 10 minutes at 3,000×g at 18° C., and the supernatant was then filtered with 0.45 filter (Sartorius, Germany). The purified CDVs and exosomes were further concentrated using Amicon 3 KDa (Millipore, US) to reach a final volume of 10 mL. The 10 mL mixture was then subjected to the size exclusion chromatography (SEC) using qEV10 (Izon Science, New Zealand). The SEC purified CDVs were further concentrated with Amicon 3 kDa and then stored at −80° C. before labeling.

CDVs and exosomes were labeled with fluorescent dye or labeled with antibodies.

After the incubation, excess dye or antibodies were removed by filtering through a centrifugal filter comprising a 300 kD molecular weight cut off, polyethersulfone filter media (NANOSEP® 300k) (antibodies) or size exclusion column (qEV10) to remove excess antibody, following the manufacturer's instruction and measure the pooled fraction at NanoFCM.

CDVs and exosomes were subjected to proteome analysis and nanoparticle flow cytometry using nanoFCM.

For single particle analysis, both CDVs and exosomes were prepped as per previous provisional patent application (use of Nanosep 300 kDa to remove excess antibody and SEC (qEV10) to remove excess CFSE.

### Results

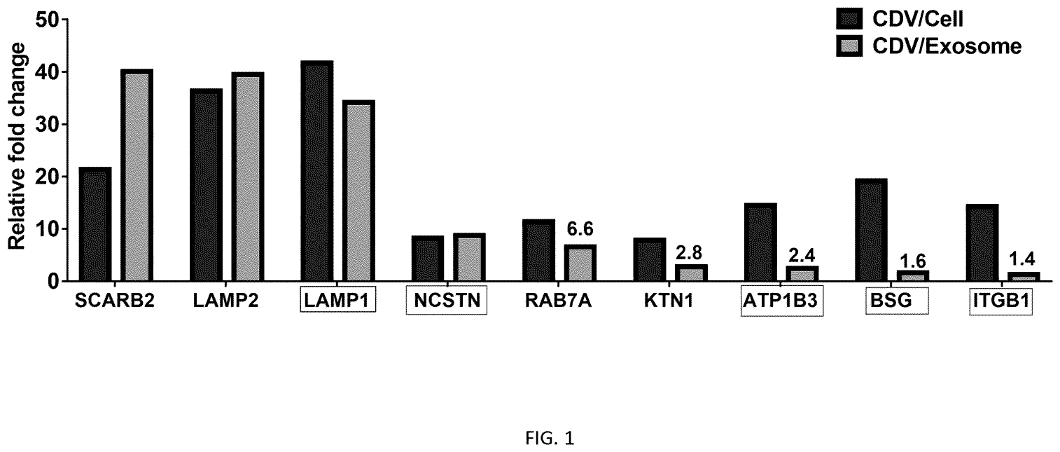

Unique CDV markers were identified relative to exosomes. Among the selected proteins, only the transmembrane proteins with fold change >5 relative to cells were identified as CDV-enriched protein markers. These CDV-enriched markers all showed higher expression than exosomes. Results are shown in FIG. 1. Abundant protein markers are highly enriched in CDVs. The expression level in CDVs was compared to cells and exosomes. SCARB2, LAMP1, and LAMP2 showed the highest expression in CDVs compared to cells and exosomes. Others selected CDV markers also had high protein abundances in CDVs relative to cells and exosomes. Protein marker in the blue box were selected for further surface marker analyses while considering the antibody availability.
SCARB2: lysosome membrane protein 2; LAMP1: lysosome-associated membrane glycoprotein 1 LAMP2: lysosome-associated membrane glycoprotein 2; NCSTN: nicastrin; RAB7A: Ras-related protein Rab-7a; KTN1: kinectin; ATP1B3: sodium/potassium transporting ATPase subunit beta-3; BSG: basigin; ITGB1: integrin beta-1.

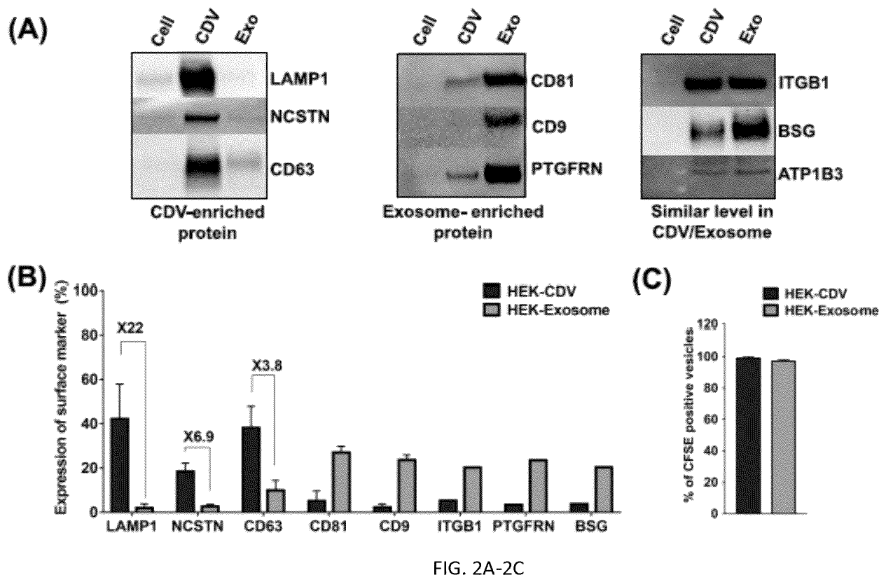

The selected membrane protein markers were analyzed by western blotting and nanoparticle flow cytometry to verify the unique CDV-specific membrane proteins identified from proteome analysis. Exosome-enriched proteins such as tetraspanin markers and Prostaglandin F2 Receptor Inhibitor (PTGFRN) were also compared. Results are shown in FIG. 2. Western blotting analyses confirmed that LAMP1, CD63, and NCSTN were enriched protein markers in CDVs. CD81, CD9, BSG, and PTGFRN were abundant in exosomes. The nanoparticle flow cytometry results were coherent with the western blotting analyses. In contrast, western blotting and nanoparticle flow cytometry results for BSG and ITGB1 did not support proteomics findings. CFSE staining revealed that more than 90% of the CDVs are intact lipid vesicles that retain membrane integrity.

### Conclusions

3 prominent CDV markers have been identified and confirmed using the purification methods described herein, indicating that CDVs are CFSE-positive intact lipid vesicles that retain membrane integrity. These findings reveal the unique mechanism of CDV biogenesis while assuring the therapeutic potential of CDVs in drug delivery. The described methods provide for more sophisticated CDV engineering that enables targeted drug delivery.

### EMBODIMENTS

Embodiment 1 is a method for processing cell-derived vesicles (CDVs), comprising: concentrating CDVs in a biological fluid; determining a concentration of the CDVs; contacting the CDVs with a fluorescent staining dye or an antibody for an CDV surface marker; incubating the contacted CDVs to generate a labeled CDV population; passing the contacted CDVs through a centrifugal filter comprising a 200-500 kD molecular weight cut off, polyethersulfone filter media, to separate the labeled CDV population from excess fluorescent staining dye or excess antibody; and recovering the labeled CDV population.

Embodiment 2 includes the method of Embodiment 1, wherein the CDVs are obtained by a method comprising preparing a suspension of nucleated mammalian cells and conducting a serial extrusion of the nucleated cells by sequentially passing them through filters with diminishing micro-size pores to produce a biological fluid comprising CDVs retaining the same membrane topology as that of the nucleated mammalian cells.

Embodiment 3 includes the method of Embodiment 2, wherein the serial extrusion comprises serially passing the nucleated mammalian cells through membrane filters with a pore size of about 10 μm, about 3 μm, and about 0.4 μm.

Embodiment 4 includes the method of Embodiment 1, wherein the concentrating comprises passing the biological fluid through a tangential flow filter.

Embodiment 5 includes the method of Embodiment 4, wherein the tangential flow filter has a molecular weight cut-off of about 300 kD to about 750 kD.

Embodiment 6 includes the method of Embodiment 1, wherein the CDVs are contacted with the fluorescent staining dye 6-Carboxyfluorescein succinimidyl ester (CFSE).

Embodiment 7 includes the method of Embodiment 1, wherein the CDVs are contacted with an anti-CD9 antibody, an anti-CD63 antibody, an anti-CD81 antibody, and/or an anti-IgG1 antibody.

Embodiment 8 includes the method of any of Embodiments 1-7, wherein the CDVs are produced from human embryonic kidney (HEK) cells, human Caucasian colon adenocarcinoma HT29 cells, mesenchymal stem cells (MSCs), embryonic stem cells and cells derived from embryonic stem cells, induced pluripotent stem cells, endothelial progenitor cells, immature and mature dendritic cells (DC), monocytes, macrophages, T and B lymphocytes, fibroblasts, epithelial cells, endothelial cells, including human umbilical vein endothelial cells (HUVEC), myocytes, cardiomyocytes, neuronal cells, glial cells, kidney cells, pancreatic cells, stromal cells, keratinocytes, or melanocytes

Embodiment 9 includes the method of any of Embodiments 1-8, wherein the concentration of CDVs in the biological fluid is determined using a flow cytometer for nanoparticle analysis.

Embodiment 10 includes the method of any of Embodiments 1-9, wherein the concentration of CDVs is determined to be at least 1×10¹⁰ CDV/ml, prior to the contacting in (c).

Embodiment 11 includes the method of any of Embodiments 1-10, wherein the contacted CDVs are passed through the centrifugal filter for at least 10 minutes at a centrifugal force of at least 10,000×g.

Embodiment 12 is a method for analyzing CDVs, comprising: concentrating CDVs in a sample with a tangential flow filter; determining a concentration of the CDVs; contacting the CDVs with a fluorescent staining dye or an antibody for a CDV surface marker; incubating the contacted CDVs to generate a labeled CDV population; passing the contacted CDVs through a centrifugal filter comprising a 300 kD molecular weight cut off, polyethersulfone filter media, to separate the labeled CDV population from excess fluorescent staining dye or excess antibody; recovering the labeled CDV population; and analyzing the recovered, labeled CDV population using a flow cytometer for nanoparticle analysis.

Embodiment 13 includes the method of Embodiment 12, wherein the CDVs are obtained by a method comprising preparing a suspension of nucleated mammalian cells, and conducting a serial extrusion of the nucleated cells by sequentially passing them through filters with diminishing micro-size pores to produce a sample comprising CDVs retaining the same membrane topology as that of the nucleated mammalian cells

Embodiment 14 includes the method of Embodiment 13, wherein the serial extrusion comprises serially passing the nucleated mammalian cells through membrane filters with a pore size of about 10 μm, about 3 μm, and about 0.4 μm.

Embodiment 15 includes the method of Embodiment 12, wherein the CDVs are contacted with the fluorescent dye 6-Carboxyfluorescein succinimidyl ester (CFSE).

Embodiment 16 includes the method of Embodiment 12, wherein the CDVs are contacted with an anti-CD9 antibody, an anti-CD63 antibody, an anti-CD81 antibody, and/or an anti-IgG1 antibody.

Embodiment 17 includes the method of any of Embodiments 12-16, wherein the CDVs are produced from human embryonic kidney (HEK) cells, human Caucasian colon adenocarcinoma HT29 cells, or mesenchymal stem cells (MSCs).

Embodiment 18 includes the method of any of Embodiments 12-17, wherein the concentration of CDVs in the sample is determined using a flow cytometer for nanoparticle analysis.

Embodiment 19 includes the method of any of Embodiments 12-18, wherein the concentration of CDVs is determined to be at least 1×10¹⁰ CDV/ml, prior to the contacting in (c).

Embodiment 20 includes the method of any of Embodiments 12-19, wherein the CDVs are concentrated using a 750 kD molecular weight cut-off tangential flow filter in (a).

Embodiment 21 includes the method of any of Embodiments 12-20, wherein the CDVs are passed through the centrifugal filter for at least 10 minutes at a centrifugal force of at least 10,000×g.

Embodiment 22 includes the method of any of Embodiments 12-21, wherein the recovered, labeled CDV population is analyzed to determine one or more of labeling efficiency, CDV number, CDV concentration, CDV protein expression, and CDV size.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A method for processing cell-derived vesicles (CDVs), comprising:
(a) concentrating CDVs in a biological fluid;
(b) determining a concentration of the CDVs;
(c) contacting the CDVs with a fluorescent staining dye or an antibody for an CDV surface marker;
(d) incubating the contacted CDVs to generate a labeled CDV population;
(e) passing the contacted CDVs through a centrifugal filter comprising a 200-500 kD molecular weight cut off, polyethersulfone filter media, to separate the labeled CDV population from excess fluorescent staining dye or excess antibody; and
(f) recovering the labeled CDV population.

2. The method of claim 1, wherein the CDVs are obtained by a method comprising preparing a suspension of nucleated mammalian cells and conducting a serial extrusion of the nucleated cells by sequentially passing them through filters with diminishing micro-size pores to produce a biological fluid comprising CDVs retaining the same membrane topology as that of the nucleated mammalian cells.

3. The method of claim 2, wherein the serial extrusion comprises serially passing the nucleated mammalian cells through membrane filters with a pore size of about 10 μm, about 3 μm, and about 0.4 μm.

4. The method of claim of claim 1, wherein the concentrating comprises passing the biological fluid through a tangential flow filter.

5. The method of claim 4, wherein the tangential flow filter has a molecular weight cut-off of about 300 kD to about 750 kD.

6. The method of claim 1, wherein the CDVs are contacted with the fluorescent staining dye 6-Carboxyfluorescein succinimidyl ester (CFSE).

7. The method of claim 1, wherein the CDVs are contacted with an anti-CD9 antibody, an anti-CD63 antibody, an anti-CD81 antibody, and/or an anti-IgG1 antibody.

8. The method of claim 1, wherein the CDVs are produced from human embryonic kidney (HEK) cells, human Caucasian colon adenocarcinoma HT29 cells, mesenchymal stem cells (MSCs), embryonic stem cells and cells derived from embryonic stem cells, induced pluripotent stem cells, endothelial progenitor cells, immature and mature dendritic cells (DC), monocytes, macrophages, T and B lymphocytes, fibroblasts, epithelial cells, endothelial cells, including human umbilical vein endothelial cells (HUVEC), myocytes, cardiomyocytes, neuronal cells, glial cells, kidney cells, pancreatic cells, stromal cells, keratinocytes, or melanocytes.

9. The method of claim 1, wherein the concentration of CDVs in the biological fluid is determined using a flow cytometer for nanoparticle analysis.

10. The method of claim 1, wherein the concentration of CDVs is determined to be at least 1×10¹⁰ CDV/ml, prior to the contacting in (c).

11. The method of claim 1, wherein the contacted CDVs are passed through the centrifugal filter for at least 10 minutes at a centrifugal force of at least 10,000×g.

12. A method for analyzing CDVs, comprising:
(a) concentrating CDVs in a sample with a tangential flow filter;
(b) determining a concentration of the CDVs;
(c) contacting the CDVs with a fluorescent staining dye or an antibody for a CDV surface marker;
(d) incubating the contacted CDVs to generate a labeled CDV population;
(e) passing the contacted CDVs through a centrifugal filter comprising a 300 kD molecular weight cut off, polyethersulfone filter media, to separate the labeled CDV population from excess fluorescent staining dye or excess antibody;
(f) recovering the labeled CDV population; and
(g) analyzing the recovered, labeled CDV population using a flow cytometer for nanoparticle analysis.

13. The method of claim 12, wherein the CDVs are obtained by a method comprising preparing a suspension of nucleated mammalian cells, and conducting a serial extrusion of the nucleated cells by sequentially passing them through filters with diminishing micro-size pores to produce a sample comprising CDVs retaining the same membrane topology as that of the nucleated mammalian cells.

14. The method of claim 13, wherein the serial extrusion comprises serially passing the nucleated mammalian cells through membrane filters with a pore size of about 10 μm, about 3 μm, and about 0.4 μm.

15. The method of claim 12, wherein the CDVs are contacted with the fluorescent dye 6-Carboxyfluorescein succinimidyl ester (CFSE).

16. The method of claim 12, wherein the CDVs are contacted with an anti-CD9 antibody, an anti-CD63 antibody, an anti-CD81 antibody, and/or an anti-IgG1 antibody.

17. The method of claim 12, wherein the CDVs are produced from human embryonic kidney (HEK) cells, human Caucasian colon adenocarcinoma HT29 cells, or mesenchymal stem cells (MSCs).

18. The method of claim 12, wherein the concentration of CDVs in the sample is determined using a flow cytometer for nanoparticle analysis.

19. The method of claim 12, wherein the concentration of CDVs is determined to be at least 1×10¹⁰ CDV/ml, prior to the contacting in (c).

20. The method of claim 12, wherein the CDVs are concentrated using a 750 kD molecular weight cut-off tangential flow filter in (a).

21. The method of claim 12, wherein the CDVs are passed through the centrifugal filter for at least 10 minutes at a centrifugal force of at least 10,000×g.

22. The method of claim 12, wherein the recovered, labeled CDV population is analyzed to determine one or more of labeling efficiency, CDV number, CDV concentration, CDV protein expression, and CDV size.
